(19) United States
(12) Patent Application Publication
(10) Pub. No.: US 2012/0053437 A1
(43) Pub. Date: Mar. 1, 2012

(54) Monitored healthcare process for the treatment of specific health problems with medical foods and/or drugs

(76) Inventor: Randall S. Hickle, Lubbock, TX (US)

(21) Appl. No.: 13/199,271

(22) Filed: Aug. 24, 2011

Related U.S. Application Data
(60) Provisional application No. 61/402,180, filed on Aug. 25, 2010.

(51) Int. Cl. A61B 5/145; G09B 19/00

(52) U.S. Cl. 600/365; 434/127

(57) ABSTRACT

A product and method for implementing and maintaining a “medical food” program for a patient having special nutrient needs in which a medical food is prescribed for said patient and for facilitating the required ongoing supervision of said patient and evaluation of the effectiveness of the prescription, the product comprising software permitting input of basic patient data, said method to include the input of basic patient information, the prescription and repeated input of physiological factors permitting review, supervision and evaluation of the effectiveness of the prescribed medical food.

### CROSS REFERENCE TO EARLIER FILED APPLICATION UNDER 35 U.S.C. §119(E)

This application is based upon and claims priority and other benefits from U.S. Provisional Patent Application Ser. No. 61/402,180 filed Aug. 25, 2010.

### FIELD OF INVENTION

This invention is directed to a low cost, highly informative and effective method of medically prescribing drugs or foods and food supplements to patients. It permits tracking of the patient's consumption of the prescribed drug, food and/or food supplement and enables cost effective supervision and evaluation by a physician or other qualified medical supervisor of the use of the prescription through the use of software and/or the internet. One focus of these disclosed inventions is to facilitate low cost, effective, documented compliance with the medical food guidelines and provisions of the Orphan Drug Act (21 U.S.C. 360ee (b) (3)) as well as the guidelines of the Office of Orphan Products Development (OOPF) within the Federal Food and Drug Administration of the U.S. Department of Health and Human Services. Requirements of those guidelines include evaluation and ongoing medical supervision of a specifically formulated and processed food product that is intended for the dietary management of the patient with special medical needs.

The stated requirement of “medical supervision” for the use of a prescribed medical food raises the immediate fears and concerns of high cost, expensive methods and procedures with exasperating, long term preventative healthcare services. This requirement also implies the usual delays and impediments of continuous, high cost visitations with physicians, of laboratory tests, of delayed evaluations, of delayed communications from busy physicians, together with undesirable waiting for information of the effectiveness of the use of the prescription. The present inventions are directed towards the elimination of these problems and the implementation of cost effective “medical supervision” of medical foods. Both patients and medical providers will find the instant inventions effective in prescribing drugs and/or medical foods, in tracking consumption, providing discipline and certainty in consumption, evaluating clinical therapeutic effects and providing early identification of potential adverse effects and enabling effective medical provider supervision in instances extending beyond medical foods.

### PRIOR ART

On belief, the prior art contains teachings of Electronic Medical Records (EMR) for patient's that are maintained on computer servers providing access to the medical providers including treating physicians as well as clinic and hospital staffs, and in some cases to billing offices. As a general rule, however, most of the data contained in those EMR's are entered and used by the Clinic and hospital staff such as the laboratory, nursing staff, physicians, etc. In some instances, patient portals may be provided, but, on belief, such portals are not intended as a means of physician supervision, disciplined consumption and evaluation. Indeed, as a general practice, it seems that the physician examines the patient, determines the needs of the patient, prescribes medications and schedules follow-up examinations to evaluate the effectiveness of the prescriptions and exercises primary control over the EMR. Such follow-up exams require time of the patient and the physician, are spaced apart to minimize time and dollar costs and provide little, if any, intervening patient supervision and/or encouragement. Indeed, in many instances, a physician may well prescribe a medication, a dietary procedure, a daily blood sugar or heart rate determination of the patient—and, without adequate supervision or discipline, the patient forgets, or ignores the prescription and/or the instruction and, consequently, the adverse patient condition continues with cost to the patient, the insurance company, the medical providers, etc. The present inventions are constructed to deal with the patient problems of forgetfulness, of lack of discipline, and when needed, lower costs of ongoing supervision and evaluation, etc.

There is also movement in the public to provide a more patient-centric electronic health record. A number of companies now offer web-based electronic medical record completely in the control of the patient. The more prominent of these companies are Google and Microsoft with Google Health (http://www.google.com/health/) and Microsoft Healthvault (http://www.healthvault.com), respectively. Both products allow the patient to organize and maintain medical information on-line and such may include information relating to prescriptions, immunizations, history of surgeries, etc. On belief, they also allow the patient to control who can access the information. Though the products allow for patient data input and information sharing, they do not intend to facilitate supervision, medical compliance by the patient, evaluation of clinical therapeutic effects and/or early identification of potential adverse effects from the medical food prescription. They largely reflect an electronic version of a filing cabinet similar to what a patient may use at home to maintain records together with limited accessibility by others.

### SUMMARY OF INVENTION

The present inventions comprise methods that provide and facilitate assurance of effective compliance with the physician's instructions and prescriptions and with the “medical food” provisions of the Orphan Drug Act and the related Code of Federal Regulations (21 CFR 101.9(j)(8). Moreover, these inventions go further and confirm compliance by the patient with the physician's instructions pertaining to the prescription, provide daily communication of that compliance to the physician or other medical provider's, facilitate continued medical supervision and further provide continuous, ongoing feedback of information pertaining to the effectiveness of the prescription—all with the intended purpose of insuring patient compliance and effective results and lack of adverse effects—while and eliminating the patient's lack of understanding, forgetfulness and/or, lack of discipline on a daily basis. These methods begin with a physician's medical examination and evaluation of the patient's needs, his prescription responding to those needs, computer program access for the patient through a patient portal as well as access by the physician and/or other medical supervisory medical providers (an Online Patient Supervisory Group), the laboratory and the pharmacy or other distribution facility which provides the medical foods.

Subject to the privacy rules of HIPPA, the methods of this invention includes entry of the lab work into the patient's portal, entry of the physician's prescriptions, the date of supply of the prescription and regular daily entry thereafter of physiological information by the patient and/or the laboratory. For example, if a physician examines an obese individual and identifies the condition of insulin resistance or pre-diabetes and prescribes a special dietary food that may also assist in preventing progression of this condition to type II diabetes, the physician may enter the prescription on the patient's medical record which is accessible by the portal, or directly into the patient portal. In addition, the laboratory might enter the blood sugar level while the pharmacist would enter the date on which the prescription was filled together with the quantity. Thereafter, the patient might confirm his consumption of the dietary medical food by date and time and also ascertain such physiological factors as weight, heart rate, blood pressures and blood sugar level as indicated by standard scales, blood pressure cuffs and blood glucose monitoring units. The patient would then regularly enter such physiological data into his patient portal for review by his physician and/or the Online Patient Supervisory/Compliance Group which is competent to routinely access the patient's portal, review the entries and ascertain the effectiveness of the prescription. Moreover, if the data is not properly entered, the physician or the staff of the Online Patient Supervisory Group may telephone the patient to provide reminders to take the prescription, and/or enter data pertaining to the physiological factors. Alternatively, the computer program may be written so as to immediately forward an email to the patient and/or the physician should the patient fail to input the desired information. Because medical foods have the risk of adverse consequences (e.g, dangerously low blood sugar, electrolyte abnormalities, and many others), the supervising physician may find it necessary to discontinue the ongoing fulfillment of the medical food prescription for noncompliant patients who do not facilitate continual medical supervision.

There are numerous medical conditions which may be identified by a physician in the process of medical evaluation which require specific dietary management due to distinctive nutritional requirements. The widespread use of specifically formulated products to treat these medically determined needs has been greatly hindered due to the absence of the invention that is the subject of this patent, an online supervisory system to satisfy the requirements of active and ongoing medical supervision. Three examples will help to elucidate the problem requiring a solution.

### Example 1

### Deficient Levels of HDL Requiring Niacin

Deficient levels of “good” cholesterol, HDL, is a common condition in this country. Medical literature establishes that deficient levels of HDL contribute greatly to the epidemic levels of heart attacks and strokes, causing the death of about ½ of all Americans. One of the most effective means of raising the levels of good cholesterol is to use a nutritional supplement, vitamin B3, also known as niacin. However, when used at doses sufficient to achieve this highly desired effect, there are risks of liver failure and muscle degeneration. Therefore, if the nutrient supplement, niacin, is to be used to the appropriate therapeutic endpoint of elevated HDL, ongoing medical supervision for the risk of adverse effects should also be provided.

### Example 2

### Inadequate Levels of Dietary EPA and DHA Requiring Supplementation

Supplements of omega-3 fatty acids have been shown to decrease the risk of heart attacks and strokes. However, these same supplements have also been associated with the increased risk of bleeding complications. Therefore, ongoing medical supervision of compliance with the appropriate dose and monitoring of the absence of adverse effects such as easy bruising are desired.

### Example 3

### Vitamin D Deficiency Treated With Vitamin D Supplementation

The condition of vitamin D deficiency has been associated with cardiovascular disease and its complications. Supplementation has been shown to be effective in raising levels. However, vitamin D is a fat soluble vitamin and therefore the body retains levels for prolonged periods of time in the fat depots of the body. Over exuberant vitamin D supplementation has been shown to cause toxic levels with the potential for severe consequences in the extreme including irreversible kidney failure which may result in death. Therefore, this supplement may be used to treat deficient levels, but ongoing medical supervision is needed to ensure safety.

Accordingly, the inventions of this application are intended to achieve, among other things, one or more of the following goals and objectives:
1) a low cost, efficient method of providing and facilitating “active and ongoing” supervision of a patient for whom a medical food has been prescribed;
2) a low cost and efficient method of continuously evaluating the use and effects of said medical foods on a patient for whom the medical food has been prescribed;
3) a low cost and efficient method of providing active and ongoing supervision of a patient who has special medically determined nutrient requirements or who has limited or impaired capacity to ingest, digest, absorb, or metabolize ordinary foodstuffs or certain nutrients; and
4) a low cost and efficient method of tracking the prescriptions, the consumption and the effects of medical food and drug prescriptions consumed by any patient.

### DESCRIPTION OF THE DRAWINGS

The manner in which the foregoing goals and objectives are achieved is disclosed in the following specification and drawings in which:

FIG. 1 is a block diagram depicting a preferred embodiment of the computer communications concept of my inventions;

FIG. 2 is a depiction of a preferred embodiment of the architecture of the overall software and hardware that may be used to implement the inventions in which the software preferably includes an HL 7 interface that may provide data from the patient's EMR if not provided directly from the external provider (e.g. physician, lab, pharmacist, etc)

FIG. 3 depicts a preferred embodiment of medical chart to be designed and programmed into said computer for immediate access by a patient, physician, medical providers and/or supervisors, laboratory personnel and pharmacists disclosing the patient's condition, prescriptions and enabling input and ongoing evaluations by the patient, the physician and/or medical providers. In this drawing, the pharmacist is also invited to record and log fulfillment of the prescriptions and laboratory personnel may also enter the results of laboratory tests for evaluation.

### DETAIL DESCRIPTION

Numerous individuals of our society have unique nutrient needs and requirements because of a limited or impaired capacity to ingest, digest, absorb, or metabolize ordinary foodstuffs. To encourage the development of foods to meet the needs of these people, Congress enacted the Orphan Drug Act (21 U.S.C. 360ee (b) (3)) and the FDA has established further criteria relating to such foods (See for example 21 CFR 101.9(J)(8). Such foods are defined as foods that are specially formulated and processed (as opposed to a naturally occurring foodstuff used in its natural state) for the patient who is seriously ill or who requires the product as a major treatment modality. Medical foods are to be prescribed by a physician as a result of a medical evaluation and are intended to be used under ongoing and active medical supervision of the patient. Such patients may include those who are or have substantial obesity, incipient type II diabetes, arthrosclerosis, Vitamin D deficiency, etc. Moreover, many patients not only suffer from one of these conditions, but often they are plagued with a combination of these conditions. For example, patients with substantial obesity often suffer from type II diabetes and/or arthrosclerosis, etc.

From an evaluation of these patients, a physician may conclude that medical foods provide an appropriate therapeutic intervention and thus prescribe the medical food that will assist the individual in eliminating, minimizing or living with the medical problem under improved circumstances. Once prescribed, however, the Orphan Drug Act requires use and/or consumption of the drug under supervision on an ongoing, active basis. That supervision raises the image of constant, recurring trips to the physician's offices by the patient. Such involves scheduling, time costs and money costs. Moreover, the physician who prescribed the medical food is left with the responsibility of supervising the use of the drug—and this supervision is dependent upon a prompt, cooperative patient.

Significantly, supervision of such matters may well focus on simple physiological measurements that can be taken at home with home instruments such as scales, blood pressure monitors, O₂ saturation meters, blood glucose monitors, etc. Alternatively, such supervision my focus on such matters as laboratory tests such as ascertainment of the patient's lipid profile, the C-Reactive Protein results, Plac2 or other test from the laboratory. Alternatively, the physician may ascertain other specific focal points to measure the effect of the medical food on the patient.

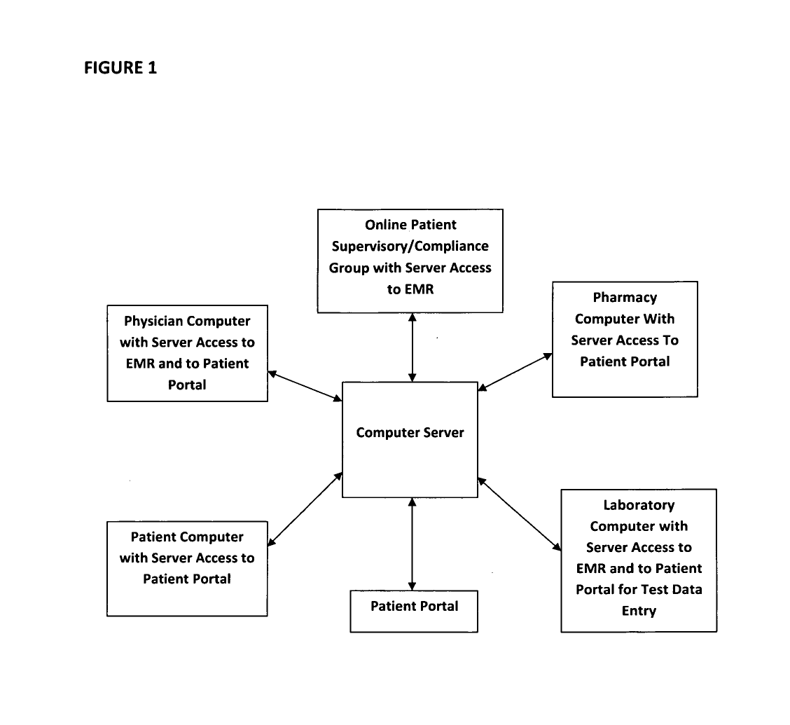

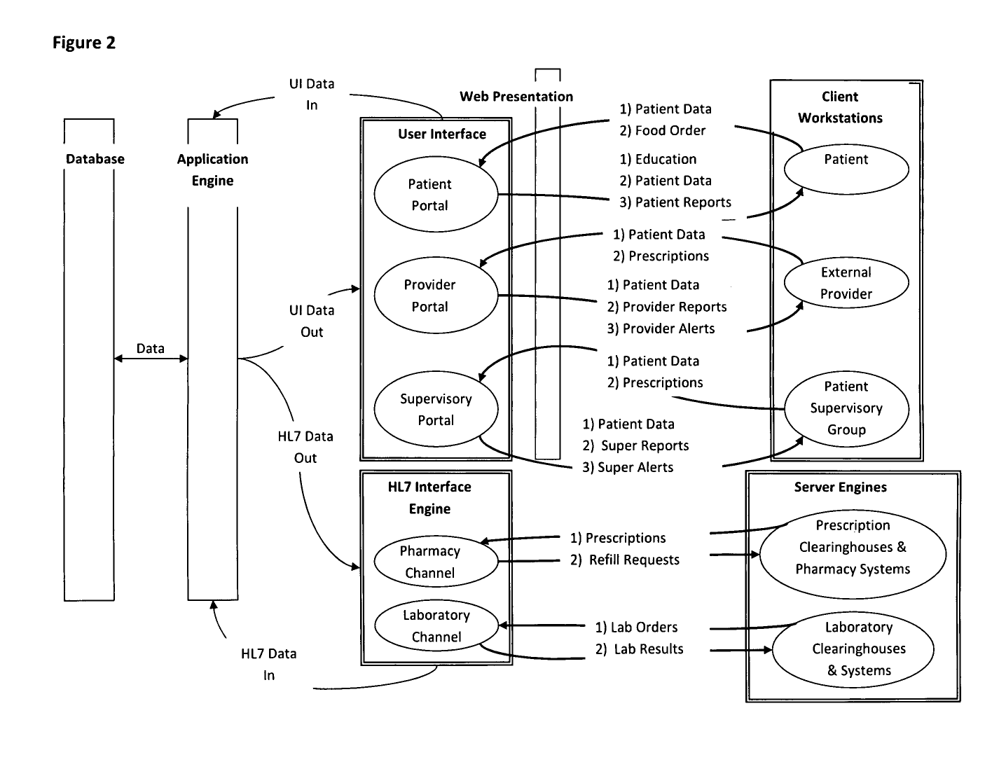

FIG. 1 discloses a block diagram for collecting and providing information and data to and from the physician, the individual patient, the laboratory, and the pharmacy and for facilitating for evaluation and supervision by the physician and/or an Online Patient Compliance Group, i.e., an online staff and, in instances, a physician. While this method of collection, evaluation and supervision is not intended as exclusive or preclusive of other needed personal observations, it does comprise a low cost, time and money efficient method of gathering, evaluating and supervising the use of the medical foods by patients. FIG. 2 depicts a preferred embodiment of the architecture that may be used in implementing the block diagrams of FIG. 1.

This method may be implemented immediately after the physician conducts an examination of the patient and makes the diagnostic determination that a medical food prescription would ameliorate the patient's condition.

At that point in time, the pertinent information is entered into a program that, preferably, is designed to run as a Patient Portal through a computer server shown in FIG. 1. In the preferred embodiment, this program permits access as well as data entry by the physician, the patient, the pharmacy, the laboratory conducting blood studies and urinalysis, and an Online Patient Supervisory/Compliance Group. In the preferred embodiment, this program would be written as a subroutine for further connectivity to different Electronic Medical Record Programs designed by various vendors and suppliers. Indeed, the Patient Portal may be designed to interact with the patient's EMR and to selectively obtain data from the EMR.

To the extent necessary and as required by HIPPA, the patient would consent to access of his patient portal by each of the entities shown in FIG. 1 through and ID and Password in case of human interaction or alternatively, secure point to point connection in the case of machine interaction. In addition, the information entered would be accessible to each.

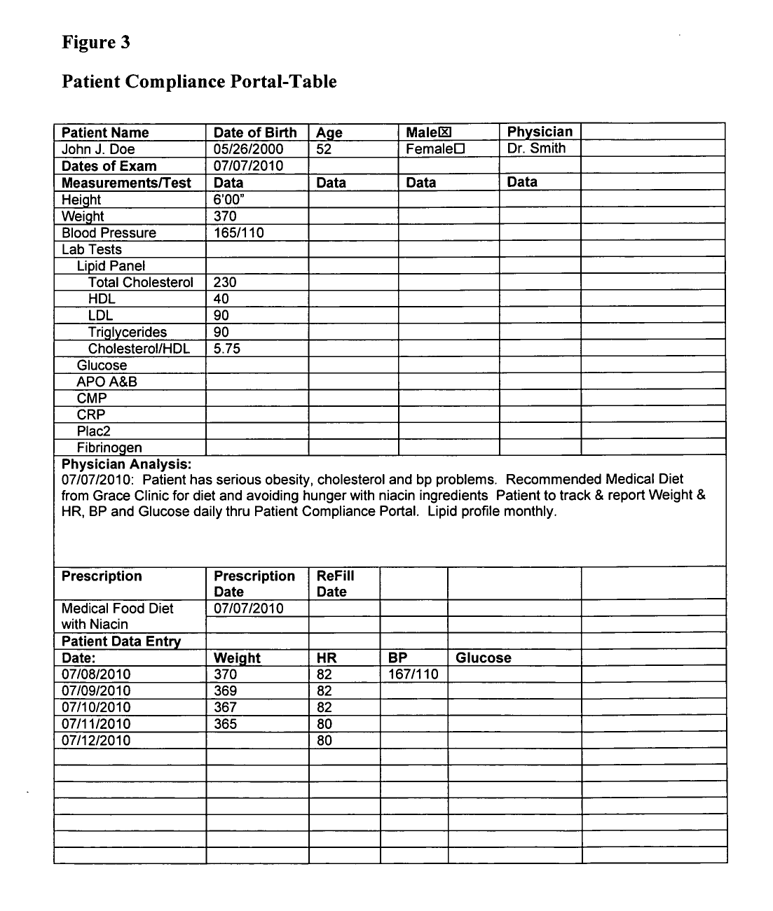

Illustrative of the information that may be maintained on this special Patient Portal is that depicted in the table of FIG. 3. It includes the patient name, DOB, age and sex together with physician identification. In addition, this table may include basic test data that is accessible to and reviewable by the patient, the provider and the Online Patient Supervisory/Compliance Center. Information from this table, in part, may be taken from the patient's EMR data base via a data importation (interactive) program or re-entered if desired. The basic test data would include selected data that assists the physician, the patient and/or the Online Patient Compliance Group in immediately ascertaining whether the patient is taking the prescription and exercising proper self-care, i.e., taking available physiological measurements and recording them at appropriate intervals, depending on the clinical requirements so as to show the effects and results of the medical food formula.

Important to the success of the medical food is proper participation by the patient. His daily interest, daily self-care, and daily data input are supervised and disciplined by the Online Patient Supervisory Group. The inventor believes this is a novel form of Medically Supervised Disciplined Self Care, the type of care which will be the most effective and cost effective of all. This Online Patient Supervisory/Compliance Center is constituted of, preferably, administrators, nurses and/or physicians. Preferably, the administrators of this group would insure compliance with the physician's instructions, the patient's monitoring and entry of data as requested by the physician. For instance, if, by a given time of the day, the patient has failed to enter his blood pressure, weight, O₂ saturation level, etc, the Center's personnel calls or emails a reminder to the patient and insists on the data entry. Alternatively, if the patient is not familiar with internet protocols and daily entry, it is anticipated that the Online Patient Supervisory/Compliance Group may operate a kiosk so as to permit those patients unfamiliar with physiological monitors and/or the interne to come to the kiosk daily and have those measurements taken with the data being entered into the form of the Patient Portal.

When the Online Supervisory/Compliance Patient Group is sufficiently large, it is anticipated RN's and MDs will monitor the Patient Portal and, in the event of health problems, they can directly call or email the patient. In addition, algorithms may well be developed to mine the patient data to ascertain those with immediate health problems and, when identified, the Supervisory Group would call the patient and advise him to seek immediate medical attention.

The invention intends to minimize manual input of data. To that end, it complies with various standards of data sharing such as Health Level Seven (HL7) for exchange of patient discrete information between disparate systems, Continuity of Care Record for importing and exporting the patient chart as defined by Healthcare Information and Management Systems Society (HIMSS), and Digital Imaging and Communications in Medicine (DICOM) for sharing medical images between disparate systems. The itemized standards are not exhaustive. They simply serve as examples of standard compliance implemented by the invention. Compliance with industry standards helps insure portability and longevity of the patient's healthcare information.

In summary, the basic functions of the software for the computer and/or server are:
1) to provide a form for each patient to accept and contain basic data concerning the patient's identification, his medical prescription, and data reflecting the physiological factors pertinent to revelation of the effectiveness of the medical prescription.
2) to permit access and entry of pertinent data on the form by the patient such as daily weight, blood pressure, heart rate, O₂ saturation levels, blood sugar levels, etc, and preferably by others such as a laboratory to record information such as the result of blood tests, etc. and/or by the pharmacy revealing the filling of the prescription, the quantity supplied and the date;
3) to permit access, patient supervision, evaluation of the data, determination of the effectiveness of the prescription as well as entry of data by the physician, and/or an on-line supervisory group for ascertaining proper entry of data by the patient and evaluation of the data entry.

Those skilled in the art will appreciate that this invention can take different forms, that the software to accomplish the functions of the inventions may take various forms, and that the table of FIG. 3 may include other entries as requested by the physician to ascertain the effectiveness of the drug. Alternatively, access might be granted to health insurers to determine, for example, the appropriateness of benefits when and if dependent upon patient compliance with the program. Various architectures of the software may be designed to accomplish the software functions described herein. Moreover, the entire program may be contained within a clinic having a kiosk for data entry by the patient, or it may be designed so that portions or all of the data can be input by the patient and various entities through the internet. Similarly, the computer software may include provisions for procurement of information through HL7, HIMSS and DICOM.

I claim:

1. A low cost method for implementing and maintaining a “medical food” program for a patient having special nutrient needs in which a medical food is prescribed for said patient and for facilitating the required ongoing supervision of said patient and evaluation of the effectiveness of the prescription, said method comprising the steps of
a) imputing patient information for the patient for whom a medical food has been prescribed into a software program of a computer
b) said program containing basic data pertaining to the patient, the patient's medical prescription, and data reflecting physiological factors pertinent to the evaluation of the effectiveness of the prescription;
c) permitting access to said program by said patient for entry of physiological data and review thereof; and
d) permitting access to one or more of the following group to permit their entry of data, review, patient supervision and/or evaluation of the effectiveness of the prescription, said group comprising the prescribing physician, pharmacy, a laboratory and an online supervisory/compliance group.

2. A method as set forth in claim 1 in which said method is directed to a patient having type II diabetic problems which comprises the further steps of:
a) using a blood glucose meter by the patient to ascertain the patient's blood glucose level at different time intervals;
b) permitting the patient to input his blood glucose level into said interactive program;
c) evaluating the effect of the medical prescription on said blood glucose level by said physician and/or supervisory compliance group.

3. A method as recited in claim 1 in which said method is conducted within a single medical facility.

4. A method as recited in claim 1 in which said method is conducted over the internet.

5. A low cost method for implementing and providing supervision and evaluation of the effectiveness of a medical food prescription by a physician, said method comprising the steps of
a) imputing patient information for the patient and said prescription into a software program of a computer
b) said program containing basic data pertaining to the patient, said prescription, and data reflecting physiological factors pertinent to the evaluation of the effectiveness of said prescription;
c) permitting access to said program by said patient for repeated entry of data pertaining to physiological conditions and review thereof; and
d) permitting access to one or more of the following group to permit their entry of data, review, patient supervision and/or evaluation of the effectiveness of said prescription, said group comprising the prescribing physician, pharmacy, a laboratory and an online supervisory/compliance group.

6. A method as recited in claim 5 in which said software program is integrated with said patient's Electronic Medical Record and said data from said software program as well as the Electronic Medical Record is available to a prescribing physician.

7. A method as recited in claim 5 in which information in said software program can be obtained from the patient's Electronic Medical Record.

8. A software product for a low cost method of implementing and providing supervision and evaluation of the effectiveness of a medical food prescription by a physician, said product comprising a software program for:
a) imputing patient information for the patient for whom said medical food has been prescribed into said software product for operation on a computer
b) said product containing basic data pertaining to the patient, said prescription, and data reflecting physiological factors pertinent to the evaluation of the effectiveness of the prescription;
c) said product permitting access by said patient for repeated entry of data pertaining to physiological conditions and review thereof; and
d) said product permitting access to one or more of the following group to permit their entry of data, review, patient supervision and/or evaluation of the effectiveness of said prescription, said group comprising the prescribing physician, pharmacy, a laboratory and an online supervisory/compliance group.

9. A product as set forth in claim 1 in which said product is directed to a patient having type II diabetic problems and in which said product accepts data pertaining to the blood glucose level of the patient.

10. A product as recited in claim 9 in which said product permits said patient to input his blood glucose level into said interactive program.

11. A product as recited in claim 10 in which said product permits viewing of the input data by the physician and his evaluation of the effectiveness of the prescription.
c) evaluating the effect of the medical prescription on said blood glucose level by said physician and/or supervisory compliance group.
